(19) United States
(12) Patent Application Publication
(10) Pub. No.: US 2024/0130861 A1
(43) Pub. Date: Apr. 25, 2024

(54) BONE IMPLANT SYSTEM, BONE IMPLANT, AND METHOD FOR PRODUCING SUCH A BONE IMPLANT

(71) Applicant: Aesculap AG, Tuttlingen (DE)

(72) Inventor: Georg Hettich, Freiburg (DE)

(21) Appl. No.: 18/278,579

(22) PCT Filed: Feb. 23, 2022
(86) PCT No.: PCT/EP2022/054544

§ 371 Date: Aug. 22, 2023

(30) Foreign Application Priority Data
Feb. 25, 2021 (DE) 10 2021 201 783.5

(51) Int. Cl. A61F 2/30

(52) U.S. Cl. CPC A61F 2/30771; A61F 2002/30115; A61F 2002/302; A61F 2002/30331; A61F 2002/30607; A61F 2002/30616; A61F 2002/4663

(57) ABSTRACT

A bone implant system, a bone implant produced using such a bone implant system, and a method for producing such an implant. The bone implant system includes a plurality of different disc elements having different external diameters. The disc elements can be stacked axially above one another in different combinations to form different stack arrangements owing to the different external diameters of the disc elements. The different stack arrangements have different outer contours. The disc elements each have an interlocking portion situated on an upper face and a complementary interlocking portion situated on an axially opposite lower face, by which interlocking portions the disc elements can be interlockingly fixed to one another in the different stack arrangements in the radial direction and/or the circumferential direction.

### CROSS-REFERENCE TO RELATED APPLICATIONS

This application is the U.S. national stage entry of International Application No. PCT/EP2022/054544, filed Feb. 23, 2022, and claims priority to German Application No. 10 2021 201 783.5, filed Feb. 25, 2021. The contents of International Application No. PCT/EP2022/054544 and German Application No. 10 2021 201 783.5 are incorporated by reference herein in their entireties.

### FIELD

The invention relates to a bone implant system, in particular for filling bone defects and/or for stabilizing a joint replacement implant. The invention also relates to a bone implant produced using such a bone implant system, and to a method for producing such a bone implant.

### BACKGROUND

Bone implants are generally known in the field of orthopedic surgery and are used, for example, in joint replacement operations in the knee, hip, shoulder and/or ankle region.

In knee replacement surgery, metaphyseal bone defects present in the proximal tibia or in the distal femur are usually treated with a bone implant. The bone implant is implanted prior to the implantation of the actual tibial or femoral joint replacement implant and is intended on the one hand to fill up the bone defect. On the other hand, the bone implant is intended to additionally stabilize the joint replacement implant in the implanted state. Corresponding bone implants are often also referred to as cones or metaphyseal cones.

To ensure successful treatment, the outer contour of the cone has to be matched to an inner contour of the bone defect that is to be treated. Cones manufactured in one piece and in different sizes and shapes are available for this purpose. However, an optimal adaptation to an existing bone defect is not possible in every case, since a suitably adapted cone cannot be manufactured and kept in stock for every conceivable defect geometry. As an alternative, individually manufactured one-piece cones are sometimes used. However, their production is time-consuming and costly.

### SUMMARY

The object of the invention is to make available a bone implant system, a bone implant and a method for producing such a bone implant, each of which partially or completely avoid the disadvantages mentioned at the outset in connection with conventional bone implants.

The bone implant system according to the invention has a plurality of different disc elements with different external diameters, wherein the disc elements are able to be stacked axially one on top of another in different combinations in order to form different stack arrangements, the different stack arrangements having different outer contours on account of the different external diameters of the disc elements, and wherein the disc elements each have a form-fitting portion arranged on an upper face and a complementary form-fitting portion arranged on an axially opposite lower face, by means of which form-fitting portions the disc elements in the different stack arrangements are able to be interlockingly fastened to one another in the radial direction and/or circumferential direction. The solution according to the invention makes it possible to dispense with time-consuming and costly production of individualized, one-piece bone implants. At the same time, however, an improved adaptation of the bone implant to a given geometry of the bone defect that is to be treated can be achieved, by comparison with conventional, non-individualized bone implants. To put it simply, depending on the geometry of the bone defect, a plurality of disc elements of the bone implant system are selected and stacked axially in combination one on top of another such that the outer contour of the resulting stack arrangement meets the requirement of matching the inner contour of the bone defect. By virtue of the different external diameters of the disc elements and the possibility of stacking the disc elements in different combinations one on top of another, it is possible to form different stack arrangements and, correspondingly, different outer contours. The form-fitting portions and the complementary form-fitting portions serve to create a form-fitting connection of the disc elements in the respectively formed stack arrangement. To put it simply, the form-fitting portions of the different disc elements are preferably designed to be the same, to be of the same type, to correspond, to be identical and/or to be interchangeable with one another. In other words, the design of the form-fitting portions is preferably independent of the external diameter of the respective disc element. The same preferably applies, mutatis mutandis, with regard to the complementary form-fitting portions. This ensures that the disc elements can always be fastened to one another, irrespective of the respectively selected combination and/or of the stack arrangement formed. For example, the plurality of disc elements can include a first disc element with a first external diameter, a second disc element with a second external diameter, and a third disc element with a third external diameter. The external diameters differ in terms of their diameter dimension and/or their shape. In this respect, the disc elements, more precisely their external diameters, can in particular be circular and/or elliptic and/or rounded in the broadest sense. Of course, the bone implant system can also have a plurality of disc elements with one and the same external diameter. For example, a plurality of first disc elements, a plurality of second disc elements and a plurality of third disc elements can be provided. The different disc elements differ at least in terms of their external diameter, in particular its dimension and/or shape. In addition, disc elements of different thickness can be provided, although these do not necessarily need to have a different external diameter at the same time. The disc elements are preferably each designed as a circular disc with a circular cross section. Preferably, the different external diameters are graded gradually in terms of their dimension. In this way, in particular, conical outer contours with different cone angles can be formed. The form-fitting portions can each have in particular a connection groove, a connection pin, a toothing or the like for form-fitting interaction with in each case one of the complementary form-fitting portions. Accordingly, the complementary form-fitting portions can each have, in particular, a feather key, a receiving bore, counter-toothing or the like. The form-fitting portions and/or the complementary form-fitting portions can be formed in one piece with the respective disc element. As an alternative, the form-fitting portions and/or the complementary form-fitting portions can be manufactured separately and then joined together with the respective disc element.

In one embodiment of the invention, the disc elements are each ring-shaped and have a preferably identical internal diameter. When stacked axially one on top of another, the internal diameters of the disc elements form a preferably circular-cylindrical receiving recess. The receiving recess is provided for receiving a shaft portion of a joint replacement implant. A circular and/or an elliptic ring shape is preferably provided.

In a further embodiment of the invention, the disc elements each have at least one spacer portion protruding in the axial direction from the upper face or the lower face, as a result of which the disc elements are able to be stacked one on top of another in the different stack arrangements while forming radial gaps. If the bone implant formed from the bone implant system is anchored in the bone defect using a connecting compound, such as bone cement for example, the radial gaps allow the connecting compound to penetrate between the individual disc elements. As a result, the stack arrangement, formed from the disc elements, or the bone implant can be additionally stabilized. In other words, the disc elements can be stacked up with gaps on account of the spacer portions that are present. The connecting compound can then pass through these gaps between the individual disc elements. The spacer portions can be formed in one piece on the respective disc element. As an alternative, the spacer portions can each be manufactured as a separate component and then joined together with the respective disc element.

In a further embodiment of the invention, the disc elements each have at least one receiving recess which is sunk into the upper face or the lower face and which is provided for receiving a connecting compound. This is especially advantageous in particular in conjunction with the features of the previous embodiment. If radial gaps are provided, the connecting compound, for example bone cement, can penetrate radially through the radial gaps between the disc elements and can be received in the receiving recesses. If there are no radial gaps, the receiving recesses can be filled with the connecting compound before the disc elements are stacked one on top of another. As a result, the formed stack arrangement/the bone implant can be inherently fixed. The receiving recesses can also be designated as receiving pockets. The receiving recesses preferably do not form a continuous connection between the upper face and lower face of the respective disc element. This is in contrast to a simple axial bore.

In a further embodiment of the invention, the form-fitting portions each have at least one axially protruding connection pin, and the complementary form-fitting portions each have at least one receiving bore introduced in the axial direction, or vice versa. The connection pins preferably protrude from the upper faces of the disc elements. The receiving bores are preferably introduced into the lower faces of the disc elements. Alternatively, the reverse arrangement can be provided. The dimensions of the connection pins and of the receiving bores are matched to each other. In the state with the disc elements interlockingly fastened to one another, the connection pins engage in the receiving bores, such that the stack arrangement formed in each case is inherently fixed in the radial and/or circumferential direction. Compared to other conceivable configurations of the form-fitting portions and/or of the complementary form-fitting portions, this embodiment of the invention allows the form-fitting connection to be produced easily and with the ability to withstand high loads. The form-fitting portions and the complementary form-fitting portions are preferably matched to one another in such a way that the disc elements can be fastened to one another in different positions of rotation relative to one another. This is preferably achieved by the fact that the number of the receiving bores is greater than the number of the connection pins. The receiving bores and/or the connection pins are preferably arranged concentrically in the radial direction.

In a further embodiment of the invention, the disc elements each have a plurality of connection pins and/or receiving bores arranged offset in the circumferential direction, the arrangements of the connection pins and/or of the receiving bores of the different disc elements forming an identical bore pattern. Put simply, this makes it possible for all the disc elements to be interlockingly connected to one another. The bore pattern of the connection pins can be characterized by the diameter of the connection pins, the diameter of a hole circle on which the plurality of connection pins of the respective disc element are arranged, a number of the connection pins per hole circle, and/or an angular position of the connection pins on the hole circle. The same applies, mutatis mutandis, with regard to the bore pattern of the receiving bores. Preferably, at least two connection pins and/or receiving bores are provided and offset by 180° from one another on the respective hole circle. Of course, more than two connection pins and/or receiving bores could also be provided. For example, three, four, five, six or more connection pins and/or receiving bores. These are preferably in each case arranged offset from one another in the circumferential direction by 120°, 90°, 72° or 60°.

In a further embodiment of the invention, at least three, preferably more than six, particularly preferably more than twelve, different disc elements are provided. These each have a different external diameter. A plurality of disc elements with one and the same external diameter can be provided, such that the bone implant system accordingly has in total more than said at least three, preferably more than six, particularly preferably more than twelve, different disc elements. If, for example, there are three disc elements of each external diameter, the bone implant system has a total of at least 9, preferably more than 18, particularly preferably more than 36 (different) disc elements.

The bone implant according to the invention has a plurality of disc elements stacked one on top of another in the axial direction with different external diameters, the disc elements stacked one on top of another being interlockingly fastened to one another in the radial direction and/or circumferential direction by means of form-fitting portions arranged on the upper face of the disc elements and complementary form-fitting portions arranged on the lower face of the disc elements. The external diameters are different in particular with regard to their dimensions and/or shape. To avoid repetition, for the advantages associated with the design of the bone implant according to the invention, reference is made to the description of the bone implant system according to the invention. What was stated there also applies, mutatis mutandis, to the bone implant according to the invention.

The method according to the invention for producing an individualized bone implant using a bone implant system according to the preceding description comprises the steps of: recording an inner contour of a bone defect; determining an outer contour of the bone implant, required to fill the bone defect, in accordance with the recorded inner contour; selecting, stacking and mutually fastening a plurality of different disc elements of the bone implant system, the plurality of disc elements being selected and/or stacked in accordance with the determined outer contour. The inner contour of the bone defect is preferably recorded intra-operatively. The inner contour can be represented by a 3D data set, for example. The recording/capturing is performed by means of a measuring technique or device, for example by means of a time-of-flight camera. The required outer contour of the bone implant is determined in accordance with the recorded inner contour. The determination is preferably computer-based. The disc elements required to represent the desired outer contour are selected by medical personnel, preferably a surgeon, from the total number of available different disc elements of the bone implant system. This is also preferably done with computer assistance, for example by means of an optimization method set up for this purpose. The selection process comprises, on the one hand, the actual selection and, on the other hand, the combining of the disc elements in the axial direction as is required to represent the desired outer contour. The selected disc elements are then stacked, preferably manually, and interlockingly fastened to one another in the radial direction and/or circumferential direction by means of the form-fitting portions and complementary form-fitting portions. Thereafter, the bone implant produced in this way can be inserted into the bone defect and anchored in the latter in a manner known in principle. The selecting of the disc elements and their assembly does not necessarily have to take place intraoperatively. Rather, the inner contour of the bone defect can be recorded in advance of an operation that is to be performed, the selecting and assembling of the disc elements being able to take place on this basis in advance of the operation.

### BRIEF DESCRIPTION OF THE DRAWINGS

Further advantages and features of the invention will become clear from the following description of preferred exemplary embodiments of the invention, which are shown in the drawings.

FIG. 1 shows a highly simplified schematic view of an embodiment of a bone implant system according to the invention having a plurality of different disc elements;

FIGS. 2, 3 and 4 show an example of a first disc element of the bone implant system according to FIG. 1 in a schematic plan view (FIG. 2), a schematic sectional view (FIG. 3) along a section line according to FIG. 2, and a schematic bottom view (FIG. 4);

FIGS. 5, 6 and 7 show an example of a second disc element of the bone implant system according to FIG. 1 in a schematic plan view (FIG. 5), a schematic sectional view (FIG. 6) along a section line VI-VI according to FIG. 5, and a schematic bottom view (FIG. 7);

FIGS. 8, 9 and 10 show an example of a third disc element of the bone implant system according to FIG. 1 in a schematic plan view (FIG. 8), a schematic sectional view (FIG. 9) along a section line IX-IX according to FIG. 8, and a schematic bottom view (FIG. 10);

FIG. 11 shows, in a sectional view rotated through 90° compared to FIGS. 3, 6 and 9, the first, second and third disc elements in a stack arrangement in which they are stacked axially one on top of another;

FIG. 12 shows the stack arrangement according to FIG. 11, the disc elements being interlockingly fastened to one another in the radial direction and/or circumferential direction;

FIG. 13 shows a highly simplified schematic view of an embodiment of a bone implant according to the invention, which is designed using the bone implant system according to FIG. 1, the bone implant being implanted proximally into a tibia in order to fill a bone defect; and

FIG. 14 shows a highly simplified schematic flow diagram illustrating an embodiment of a method according to the invention for producing a bone implant according to the invention.

### DETAILED DESCRIPTION

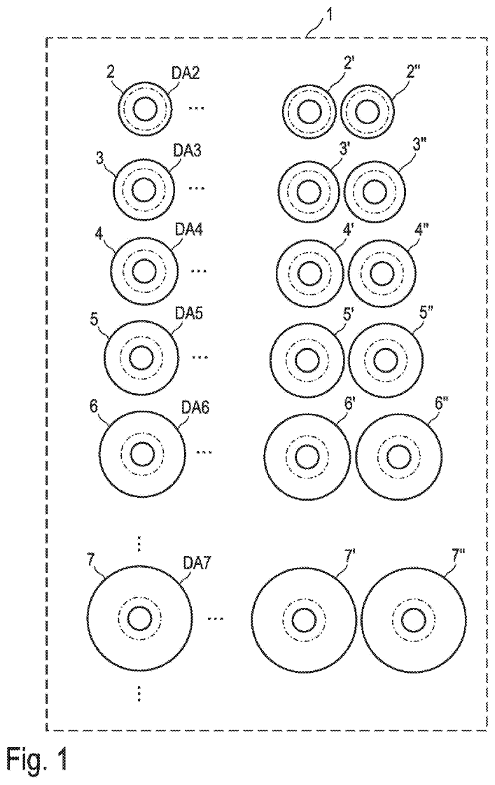

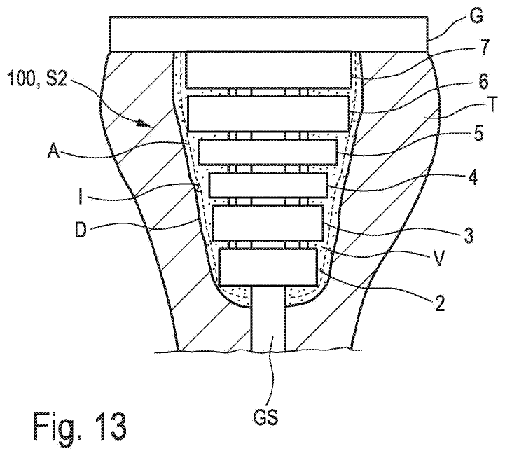

According to FIG. 1, a bone implant system 1 is provided for forming an individualized bone implant 100 (FIG. 13). In the embodiment shown, the bone implant 100 is provided for use in a knee joint replacement operation and is shown by way of example in an implanted state in FIG. 13. In this implanted state, the bone implant 100 is implanted proximally in a tibia T. In the use shown here, the bone implant 100 primarily fulfils two functions. On the one hand, the bone implant 100 fills a metaphyseal bone defect D in the region of the proximal tibia T. On the other hand, the bone implant 100 stabilizes a tibial joint replacement implant G. In the present case, the joint replacement implant G is what is called a tibial plateau, which is anchored proximally to the tibia T, in a manner known to those skilled in the art, and is set up to interact with further components of a knee joint replacement prosthesis. To achieve optimal treatment results, it is desirable for an outer contour A of the bone implant 100 to be geometrically matched to an inner contour I of the bone defect D. The outer contour A is shown in greatly simplified form in dashed lines in FIG. 13 and is offset approximately parallel to the inner contour I.

Metaphyseal bone defects can naturally have a wide variety of geometries. However, in order to allow the best possible adaptation to the respective inner contour, individualized bone implants are known in the prior art. After the geometry of the bone defect has been determined, these implants are individually manufactured in one piece as a so-called “monobloc”. Such custom-made products can be time-consuming and expensive. In addition, bone implants specifically designated for the present use as tibia cones are commercially available in different sizes and shapes. An improved adaptation to the respective inner contour of the bone defect is intended to be achieved through a suitable selection of the size and/or shape of the bone implant. However, this is not successful in every case.

The bone implant system 1 overcomes the disadvantages associated with the prior art and in particular permits time-saving and cost-saving production of individualized bone implants that are matched to an existing defect geometry. The bone implant system 1 is preferably suitable for the use described above in the context of a knee joint replacement operation. Besides this, the bone implants that can be produced by means of the bone implant system 1 can also be used in other joint replacement operations, for example in the hip, shoulder and/or ankle region.

The bone implant system 1 has a plurality of different disc elements 2 to 7 with different external diameters DA2 to DA7 (FIG. 1).

FIG. 1 shows examples of six disc elements that differ in terms of their outer diameter. It will be appreciated that the bone implant system can have fewer than the six different disc elements shown, for example two, three, four or five, or more than the six different ones shown, for example seven, eight, nine or ten. This is symbolized in FIG. 1 by the dots marked between the disc elements 6 and 7 and below the disc element 7.

The size relationships between the different external diameters DA2 to DA7 that can be seen in FIG. 1 are to be understood purely as examples. It will be appreciated that, in embodiments not shown in the drawings, the different external diameters can have a finer or broader graduation.

In addition, in the embodiment shown, the bone implant system 1 has a plurality of identical disc elements for each external diameter DA2 to DA7. In this respect, it is possible to have a first set of disc elements 2, 2′, 2″ with the external diameter DA2, a second set of disc elements 3, 3′, 3″ with the external diameter DA3, a third set of disc elements 4, 4′, 4″ with the external diameter DA4, etc. FIG. 1 shows an example with three disc elements per set. In an embodiment not shown in the drawings, only one disc element is provided for each external diameter. In further embodiments not shown, two, four, five, six or more disc elements are provided per set, for example. Against this background, the disc elements 2, 2′, 2″ can each also be referred to as first disc element. Accordingly, the disc elements 3, 3′, 3″ can each be referred to as second disc element. The disc elements 4, 4′, 4″ can each be referred to as third disc element. The further disc elements of the bone implant system 1 are designated accordingly. For the sake of brevity, only the first disc element 2, the second disc element 3, the third disc element 4, the fourth disc element 5, etc., will be discussed below.

In the embodiment shown, the different external diameters DA2 to DA7 differ only in terms of their dimensions. It will be appreciated that the different external diameters DA2 to DA7 can alternatively or additionally differ in terms of their shape. In this respect, the circular or annular design of the external diameters shown in the present figures is to be understood purely as an example and is attributable to the simplified graphical representation. Instead of the shape shown in the drawings, the different external diameters DA2 to DA7 can in particular be elliptic and/or rounded in the broadest sense. For example, an elliptic shape can also be used to treat asymmetrical bone defects.

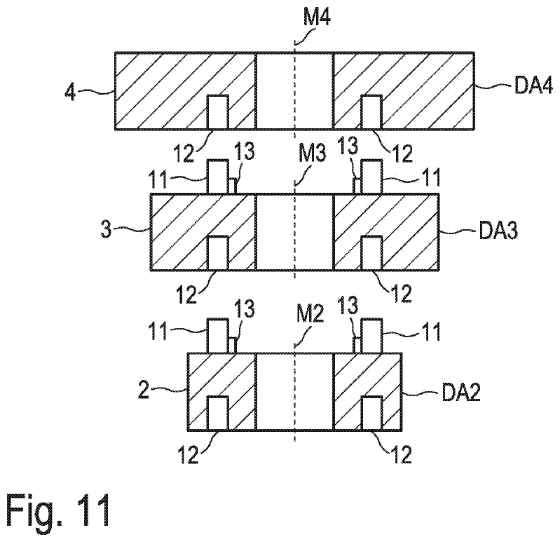

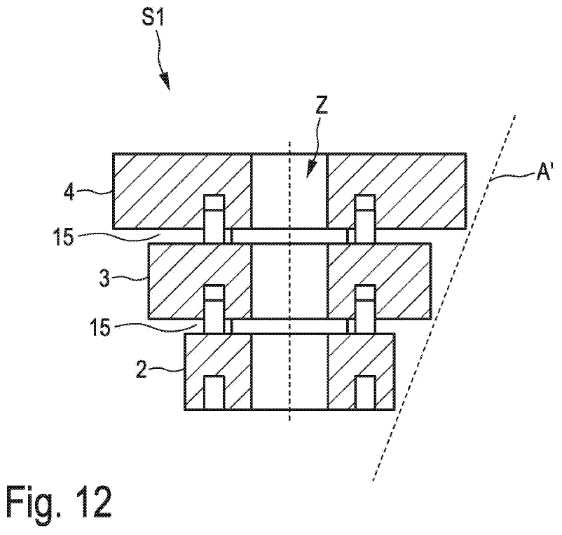

The different disc elements 2 to 7 are able to be stacked axially one on top of another in different combinations to form different stack arrangements. An example of a first stack arrangement S1 is shown in FIGS. 11 and 12. The stack arrangement S1 is formed, for example, by one of the first, one of the second and one of the third disc elements or by the first disc element 2, the second disc element 3 and the third disc element 4. On account of the different external diameters DA2, DA3, DA4 and the specially chosen sequence of the disc elements 2, 3, 4 in the axial direction, the stack arrangement S1 has an outer contour A′. This is shown schematically in a highly simplified manner in FIG. 12 and is conical in the broadest sense. In contrast to this, the bone implant 100 is formed by a different, further stack arrangement S2 of disc elements of the bone implant system 1, which will be described in greater detail, and has the outer contour A already mentioned.

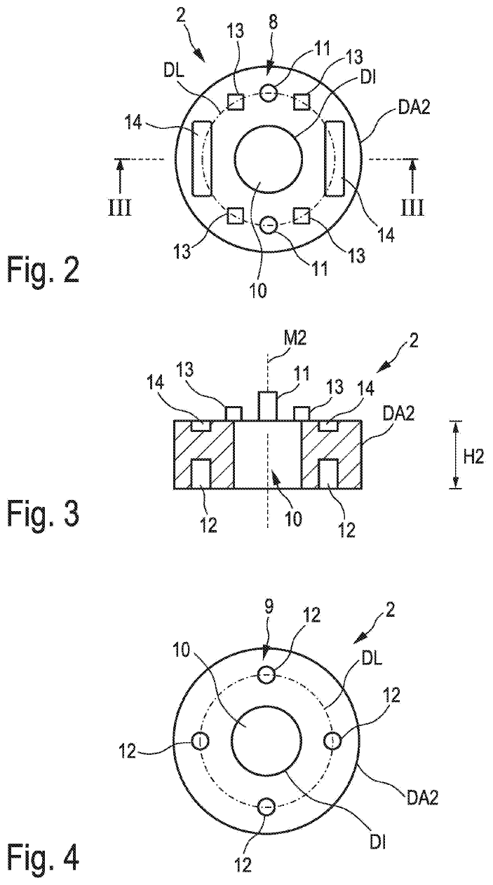

Depending on the selection and stacking sequence of the different disc elements 2 to 7 of the bone implant system 1, a wide variety of stack arrangements and thus bone implants with the most varied of outer contours can be formed. The disc elements in question are not just placed loosely one on top of another, but interlockingly fastened to one another in the radial direction and circumferential direction. For this purpose, the different disc elements 2 to 7 each have a form-fitting portion 8 and a complementary form-fitting portion 9 (cf. in particular FIGS. 2 to 10). The design and functioning of the form-fitting portions 8 and of the complementary form-fitting portions 9 and also the remaining design and functioning of the different disc elements 2 to 7 are explained below with reference to FIGS. 2 to 10 as examples with regard to the first disc element 2, the second disc element 3 and the third disc element 4.

In the embodiment shown, the first disc element 2 (FIGS. 2 to 4) is designed in the shape of a circular ring and can therefore also be referred to as a circular ring element. By virtue of the ring-shaped design, the disc element 2 has an internal diameter DI. The internal diameter DI is formed by a central bore 10 which extends coaxially with respect to a central longitudinal axis M2 of the first disc element 2. In the present case, the (first) external diameter DA2 is concentric with respect to the internal diameter DI. The first disc element 2 has an axial thickness H2. In an embodiment not shown in the drawings, the disc elements are elliptic.

The form-fitting portion 8 is arranged on an upper face (FIG. 2) of the first disc element 2. The complementary form-fitting portion 9 is arranged on an axially opposite lower face (FIG. 4).

In the embodiment shown, the form-fitting portion 8 has two connection pins 11 which are offset by 180° about the central longitudinal axis M2. The connection pins 11 protrude axially from the upper face and have a circular-cylindrical solid cross section.

In an embodiment not shown in the drawings, the form-fitting portion can have just one connection pin or more than the two connection pins shown here.

The connection pins 11 can be formed in one piece on the upper face of the first disc element 2 or can be manufactured as separate components and joined to the upper face. In the embodiment shown, the connection pins are separate components and are each inserted into receiving bores, not designated in any further detail, on the upper face of the first disc element 2. In the present case, the complementary form-fitting portion 9 has four receiving bores 12 which are offset in each case by 90° about the central longitudinal axis M2. The receiving bores 12 extend parallel to the central longitudinal axis M2. The same also applies to the connection pins 11.

In an embodiment not shown in the drawings, the complementary form-fitting portion 9 has fewer or more than the four receiving bores 12 shown here.

The connection pins 12 are arranged on a hole circle with a hole circle diameter DL. The same applies to the connection pins 11. The hole circle diameter DL or the hole circle is shown in dot-and-dash lines in FIGS. 2 and 4.

In the embodiment shown, the hole circle is concentric with respect to the central longitudinal axis M2. In the present case, concentricity is therefore also present in relation to the internal diameter DI and the first external diameter DA2.

Furthermore, the first disc element 2 in the present case has at least one spacer portion 13 which protrudes in the axial direction from the upper face. In the present case, a total of four spacer portions 13 are provided and are each arranged in pairs adjacent to the connection pins 11 in the region of the hole circle diameter DL. In the embodiment shown, the spacer portions 13 have a square cross section, which is to be understood purely by way of example. The spacer portions 13 can be formed in one piece on the upper face or can be connected as separate components to the upper face of the first disc element 2. In the embodiment shown, the spacer portions 13 are each designed as separate spacer pins and, in a manner not shown in detail, are inserted into bores made in the upper face of the first disc element 2.

In an embodiment not shown in the drawings, the spacer portions are arranged on the lower face. In addition, it will be appreciated that the spacer portions do not necessarily have to be arranged in the region of the hole circle diameter DL.

In the present case, the first disc element 2 also has two receiving recesses 14 which are axially sunk into the upper face. The receiving recesses 14 can also be designated as receiving pockets. The two receiving recesses 14 are offset from each other by 180° with respect to the central longitudinal axis M2 and each have a rectangular basic shape. The arrangement and the design of the receiving recesses are both to be regarded purely as examples.

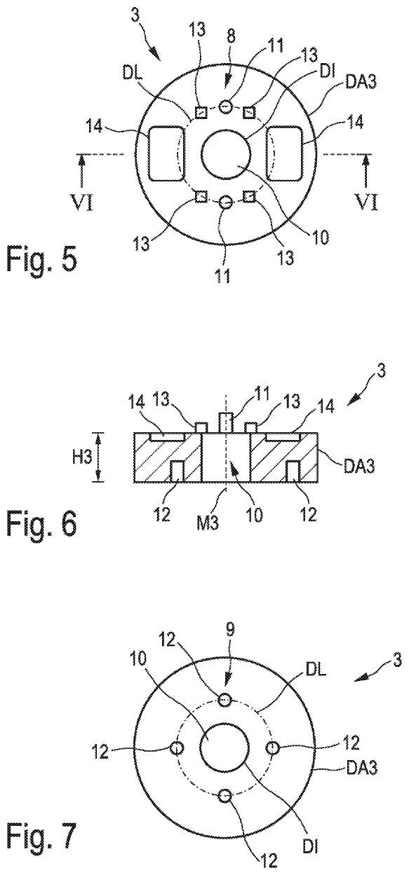

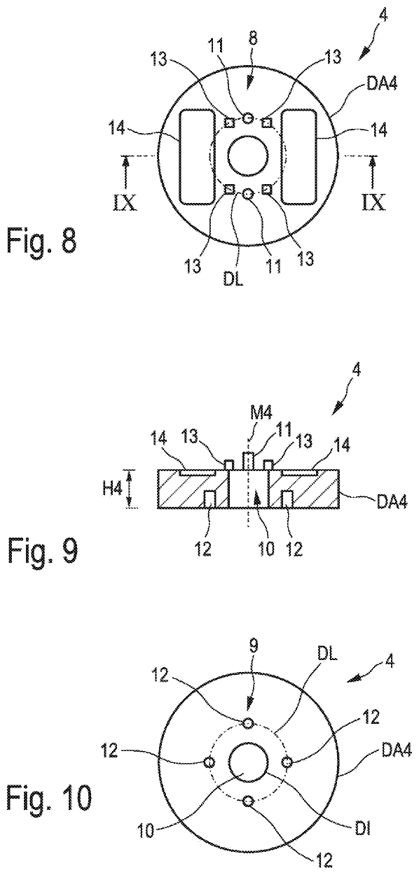

The second disc element 3 (FIGS. 5 to 7) and the third disc element 4 (FIGS. 8 to 10) are identical to the first disc element 2 in terms of the design and/or arrangement of the form-fitting portion 8 and of the complementary form-fitting portion 9. Identical components and/or parts are accordingly given identical reference numbers. What has been said concerning the form-fitting portion 8 and the complementary form-fitting portion 9 in connection with the first disc element 2 thus applies, mutatis mutandis, to the second disc element 3 and the third disc element 4 and moreover to all other disc elements of the bone implant system 1. By virtue of the identical design and/or arrangement of the form-fitting portions and of the complementary form-fitting portions, it is ensured, to put it in simple terms, that all the disc elements of the bone implant system 1 can be fastened to one another independently of the respective stack arrangement.

The design and arrangement of the spacer portions 13 is also identical. This is advantageous, but not essential. In an embodiment not shown, the spacer portions of the different disc elements 2 to 7 can be designed and/or arranged differently.

Furthermore, like the first disc element 2, the second disc element 3 also has receiving recesses 14 sunk into the upper face. The receiving recesses 14 of the second disc element 3 differ only in terms of their size compared to the receiving recesses 14 of the first disc element 2, and therefore a separate reference number is not used. The same applies, mutatis mutandis, with regard to the third disc element 4 and its receiving recesses 14.

Moreover, the second disc element 3 has an axial thickness H3. The third disc element 4 has an axial thickness H4. In the embodiment shown, the first, second and third disc elements are of the same thickness, such that the thicknesses H2, H3 and H4 match. In an embodiment not shown in the drawings, different disc elements have different thicknesses. In addition, it is conceivable that disc elements with one and the same external diameter have different thicknesses. For example, within the first set of disc elements 2, 2′, 2″ and/or the remaining sets of disc elements of the bone implant system 1.

The first disc element 2, or one of the first disc elements 2, 2′, 2″, is selected in order to form the stack arrangement S1 shown in FIG. 12. In addition, the second disc element 3 and the third disc element 4 or one of the respective disc elements are selected. The second disc element 3 is oriented in axial alignment with the first disc element 2, such that the central longitudinal axes M2, M3 are oriented coaxially. In addition, the second disc element 3 is positioned in the circumferential direction relative to the first disc element 2 in such a way that the connection pins 11 of the latter can engage in the receiving bores 12 of the second disc element 3. Thereafter, the second disc element 3 is plugged in the axial direction onto the first disc element 2. In this way, a form fit between the connection pins 11 and the receiving bores 12 is formed both radially and in the circumferential direction. On account of the spacer portions 13 arranged on the upper face of the first disc element 2, a radial gap 15 extending in the axial direction is obtained (FIG. 12). Thereafter, the third disc element 4 is stacked onto the second disc element 3, with the central longitudinal axes M3, M4 being oriented coaxially. This in turn creates a corresponding form fit between the connection pins 11 of the second disc element 3 and the receiving bores 12 of the third disc element 4. In addition, a radial gap 15 is also obtained between the second disc element 3 and the third disc element 4.

With regard to FIGS. 11 and 12, it is worth noting that the connection pins 11 and also the spacer portions 13 of the third disc element 4 can be removed, by virtue of their being designed as separate components. This is advantageous but not essential.

In the stack arrangement S1, the central bores 10 or the internal diameters DI form a cylindrical receiving recess Z. The receiving recess Z is suitable for receiving a shaft portion GS of the joint replacement implant G (FIG. 13).

The axial gaps 15 formed between the disc elements 2, 3, 4 allow a connecting compound V to penetrate and/or be introduced (cf. FIG. 13). As a result, the stack arrangement S1 can be additionally stabilized. Alternatively or additionally, the connecting compound V can be introduced into the receiving recesses 14 before the disc elements 2, 3, 4 are stacked one on top of another.

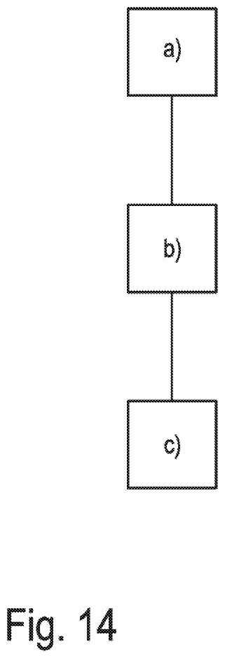

An embodiment of a method according to the invention for producing the bone implant 100 using the bone implant system 1 is illustrated schematically in FIG. 14. The method comprises steps a), b) and c).

In step a), the inner contour I of the metaphyseal bone defect D is first of all recorded by measurement. Measuring methods suitable for this purpose are known in principle to a person skilled in the art. For example, a time-of-flight camera can be used.

In step b), the outer contour A of the bone implant 100 required for filling the bone defect D is determined. In this case, the outer contour A is determined in accordance with the previously recorded inner contour I, preferably with the aid of a computer and/or based on simulation.

In step c), a plurality of different disc elements of the bone implant system 1 are selected, stacked and fastened to one another. The plurality of disc elements are selected and/or stacked in accordance with the determined outer contour A, preferably during surgery. The selection is made by medical personnel, preferably with the aid of a computer and/or based on simulation. In the present case, the or a first disc element 2, a second disc element 3, a third disc element 4, a fourth disc element 5, a fifth disc element 6 and a sixth disc element 7 are selected and stacked one on top of another. Said disc elements are connected by means of the form-fitting portions 8 and the complementary form-fitting portions 9, as has previously been described. FIG. 13 shows individual disc elements 2 to 7 with different thicknesses. This is intended to illustrate once again that not all disc elements of the bone implant system 1 necessarily need to have one and the same axial thickness. After the disc elements 2 to 7 have been manually stacked one on top of another and interlockingly fastened to one another, the bone implant 100 can be inserted into the bone defect D and anchored there by means of the connecting compound V, which is a bone cement in the present case. The connecting compound V can be introduced into the cylindrical receiving recess Z or between the different disc elements 2 to 7 before the bone implant 100 is inserted. This achieves additional stabilization of the bone implant 100. After the bone implant 100 has been anchored, the joint replacement implant G can be attached proximally to the tibia T. Here, the shaft portion GS is inserted axially into the cylindrical receiving recess Z and is anchored therein in the radial direction.

1. A bone implant system comprising:
a plurality of disc elements with different external diameters,
wherein the disc elements are axially stackable one on top of another in different combinations in order to form different stack arrangements, the different stack arrangements having different outer contours on account of the different external diameters of the disc elements, and
wherein the disc elements each have a form-fitting portion arranged on an upper face and a complementary form-fitting portion arranged on a lower face that is axially opposite the upper face, by which form-fitting portions the disc elements in the different stack arrangements are interlockingly fastenable to one another in a radial direction and/or a circumferential direction.

2. The bone implant system according to claim 1, wherein the disc elements are each ring-shaped and have an internal diameter.

3. The bone implant system, according to claim 1, wherein the disc elements each have at least one spacer portion protruding in an axial direction from the upper face or the lower face, as a result of which the disc elements are stackable one on top of another in the different stack arrangements while forming axial gaps.

4. The bone implant system according to claim 1, wherein the disc elements each have at least one receiving recess which is sunk into the upper face or the lower face and which is provided for receiving a connecting compound.

5. The bone implant system according to claim 1, wherein the form-fitting portions each have at least one axially protruding connection pin, and wherein the complementary form-fitting portions each have at least one receiving bore introduced in the axial direction, or vice versa.

6. The bone implant system according to claim 5, wherein the disc elements each have a plurality of connection pins and/or receiving bores arranged offset in the circumferential direction, the arrangements of the connection pins and/or the receiving bores of the disc elements forming an identical bore pattern.

7. The bone implant system according to claim 1, wherein the disc elements comprise at least three disc elements.

8. A bone implant comprising a plurality of disc elements stacked one on top of another in an axial direction with different external diameters, the disc elements stacked one on top of another being interlockingly fastened to one another in a radial direction and/or a circumferential direction by means of form-fitting portions arranged on an upper face of the disc elements and complementary form-fitting portions arranged on a lower face of the disc elements.

9. A method for producing a bone implant using a bone implant system according to claim 1, comprising the steps of:
a) recording an inner contour of a bone defect;
b) determining an outer contour of the bone implant required to fill the bone defect, in accordance with the inner contour;
c) selecting, stacking and mutually fastening a plurality of different disc elements of the bone implant system, the plurality of disc elements being selected and/or stacked in accordance with the outer contour.
